(19) United States
(12) Patent Application Publication
(10) Pub. No.: US 2008/0172103 A1
(43) Pub. Date: Jul. 17, 2008

(54) METHODS AND SYSTEM FOR BRAIN STIMULATION

(75) Inventors: Changquing Chris KAO, Brentwood, TN (US); Peter E. KONRAD, Franklin, TN (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: VANDERBILT UNIVERSITY, Nashville, TN (US)

(21) Appl. No.: 12/015,892

(22) Filed: Jan. 17, 2008

Related U.S. Application Data
(60) Provisional application No. 60/880,846, filed on Jan. 17, 2007.

(51) Int. Cl. A61N 1/05

(52) U.S. Cl. 607/45

(57) ABSTRACT

A method for stimulating a target of interest of a living subject with reduction of power consumption. In one embodiment, the method includes the steps of delivering a plurality of pulses to the target of interest in a substantially repeating pattern for a first period of time, T₁, which is immediately followed by a second period of time, T₂, during which no pulses are delivered to the target of interest, wherein the plurality of pulses is delivered such that any two neighboring pulses of the plurality of pulses are occurred in a third period of time, T₃; and repeating the delivering step for a predetermined times, where T₁ and T₂ are in the order of milliseconds, and T₁>T₃ and T₂≧T₃.

### CROSS-REFERENCE TO RELATED PATENT APPLICATION

This application claims the benefit, pursuant to 35 U.S.C. §19(e), of U.S. provisional patent application Ser. No. 60/880,846, filed Jan. 17, 2007, entitled “METHODS AND SYSTEM FOR BRAIN STIMULATION,” by Changquing Chris Kao, and Peter E. Konrad, which is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [2] represents the second reference cited in the reference list, namely, B. Schrader, W. Hamel, D. Weinert, and H. M. Mehdorn, “Documentation of electrode localization.” Movement Disorders, vol. 17 (supplement 3), pp S167-S174, 2002.

### FIELD OF THE INVENTION

The present invention relates generally to stimulation, and more particularly to methods and systems that utilize a train stimulation of a target of interest of a living subject with reduction of power consumption.

### BACKGROUND OF THE INVENTION

Since its first Food and Drug Administration (FDA) approval in 1998, deep-brain stimulation (DBS) has gained significant popularity in the treatment of a variety of brain-controlled disorders, including movement disorders [1, 2]. The therapy of the DBS has significant applications in the treatment of tremor, rigidity, and drug induced side effects in patients with Parkinson's disease and essential tremor. Generally, such treatment involves placement of one or more DBS electrode leads in areas including the subthalamic nucleus (STN) and/or the ventralis intermedius nucleus (VIM) of the thalamus of the brain of a patient through one or more burr holes drilled in the patient's skull, followed by placement of the one or more electrode leads and then applying appropriate stimulation signals through the one or more electrode leads to the physiological target. The one or more electrode leads are coupled to a pulse generator that is implanted under the skin of the patient. The placement procedures of the treatment, involving stereotactic neurosurgical methodology, are very sophisticated, time-consuming and costly.

Electrical stimulations of other anatomical regions of a patient may be used to control pain or to treat other disorders. For example, application of an electrical field to spinal nervous tissue can effectively mask certain types of pain transmitted from regions of the body associated with the stimulated tissue. In general, the electrical stimulation is delivered to a target of interest with a stimulation device having one or more electrode leads implanted in the target of interest and a pulse generator coupled to one or more electrode leads for generating appropriate stimulation signals.

In operation, the patient may use a hand-held magnet or other means to turn the pulse generator on or off. The pulse generator produces high-frequency stimulation signals that are delivered to the target of interest by the one or more electrode leads for stimulation thereof.

Usually, these stimulation devices have a limited power source such as a battery and require periodic services or replacements. For example, the battery of a stimulation device must be replaced when it no longer supplies adequate power to the pulse generator for generating appropriate stimulation signals. The time until the pulse generator needs to be replaced is dependent, in part, on the operation time and pulse characteristics of the pulse generator. For a DBS, implanted stimulators typically require battery replacement every three to five years. Such a battery replacement involves time-consuming and costly surgical procedures. On the other hand, allowing the battery to deplete itself to a level that the pulse generator can no longer provide adequate therapy, or stops working altogether, can be problematic for the patient.

Therefore, a heretofore unaddressed need exists in the art to address the aforementioned deficiencies and inadequacies.

### SUMMARY OF THE INVENTION

The present invention, in one aspect, relates to a method for reducing power consumption in an implantable stimulation device having an internal pulse generator (IPG), a power supply adapted for powering the IPG, and at least one electrode operably coupled with the IPG.

In one embodiment, the method includes the steps of causing the IPG to generate a train of electrical pulses, where the train of electrical pulses comprises a series of pulse sets, each of the plurality of pulse sets having a plurality of pulses time-evenly distributed over a first period of time, T₁, any two neighboring pulse sets of the series of pulse sets being separated by a second period of time, T₂, and any two neighboring pulses of the plurality of pulses being separated by a third period of time, T₃; and delivering the train of electrical pulses to a target of interest of a living subject for stimulation by the at least one electrode placed in the target of interest. The target of interest of the living subject is corresponding to the STN, or the VIM of the thalamus of the brain of the living subject.

In one embodiment, the implantable stimulation device further comprises a controller being operable to cause the IPG to generate the train of electrical pulses.

The first period of time T₁ and the second period of time T₂ are in the order of milliseconds, and wherein T₁>T₃ and T₂≧T₃. In one embodiment, 0.3<(T₂/T₁)<0.8. The first period of time T₁ is in the range of about 80-120 ms, and the second period of time T₂ is in the range of about 30-50 ms.

The plurality of pulses is characterized with a pulse width, τ, an amplitude, H, and a frequency, f=1/T, wherein T=τ+T₃ being a pulse period. In one embodiment, the frequency f is in the range of about 2-1000 Hz.

Furthermore, the method includes the step of determining the pulse width τ, the amplitude H, and the frequency f of the plurality of pulses, the first period of time T₁ and the second period of time T₂. In one embodiment, the determining step comprises the steps of: delivering an electrical signal having pulses in a substantially repeating pattern to the target of interest for a continuous stimulation of the target of interest, wherein the electrical signal is characterized with a pulse width, τ₀, an amplitude, H₀, and a frequency, f₀; adjusting the pulse width τ₀, the amplitude H₀, and the frequency f₀ of the electrical signal so as to obtain an optimal efficacy of the continuous stimulation of the target of interest; delivering a train of electrical pulses to the target of interest for a train stimulation of the target of interest, wherein the train of electrical pulses comprises a series of pulse sets, each pulse sets having a plurality of pulses with a pulse width τ=τ₀, an amplitude H=H₀, and a frequency f=f₀, time-evenly distributed over a first period of time, T₁, and any two neighboring pulse sets being separated by a second period of time, T₂; and adjusting the first period of time T₁ and the second period of time T₂ of the train of electrical pulses so that the efficacy of the train stimulation is identical to the optimal efficacy of the continuous stimulation of the target of interest.

In another aspect, the present invention relates to a method for stimulating a target of interest of a living subject with a stimulation device implanted therein, the stimulation device having an IPG, a power supply adapted for powering the IPG, and at least one electrode placed in the target of interest and operably coupled with the IPG. The target of interest of the living subject is corresponding to the STN, or the VIM of the thalamus of the brain of the living subject.

In one embodiment, the method includes the step of causing the IPG to generate a train of electrical pulses. The train of electrical pulses comprises a series of pulse sets, where each of the plurality of pulse sets has a plurality of pulses time-evenly distributed over a first period of time, T₁, any two neighboring pulse sets of the series of pulse sets are separated by a second period of time, T₂, and any two neighboring pulses of the plurality of pulses are separated by a third period of time, T₃. The plurality of pulses is characterized with a pulse width, τ, an amplitude, H, and a frequency, f=1/T, wherein T=τ+T₃ being a pulse period. In one embodiment, the frequency f is in the range of about 2-1000 Hz. In one embodiment, the first period of time T₁ and the second period of time T₂ are in the order of milliseconds, and wherein T₁>T₃ and T₂≧T₃. In one embodiment, 0.3<(T₂/T₁)<0.8. The first period of time T₁ is in the range of about 80-120 ms, and the second period of time T₂ is in the range of about 30-50 ms.

In one embodiment, the implantable stimulation device further comprises a controller being operable to cause the IPG to generate the train of electrical pulses.

Furthermore, the method includes the step of delivering the train of electrical pulses to a target of interest of a living subject for stimulation by the at least one electrode.

Additionally, the method also includes the step of determining the pulse width τ, the amplitude H, and the frequency f of the plurality of pulses, the first period of time T₁ and the second period of time T₂. In one embodiment, the determining step comprises the steps of: delivering an electrical signal having pulses in a substantially repeating pattern to the target of interest for a continuous stimulation of the target of interest, wherein the electrical signal is characterized with a pulse width, τ₀, an amplitude, H₀, and a frequency, f₀; adjusting the pulse width τ₀, the amplitude H₀, and the frequency f₀ of the electrical signal so as to obtain an optimal efficacy of the continuous stimulation of the target of interest; delivering a train of electrical pulses to the target of interest for a train stimulation of the target of interest, wherein the train of electrical pulses comprises a series of pulse sets, each pulse sets having a plurality of pulses with a pulse width τ=τ₀, an amplitude H=H₀, and a frequency f=f₀, time-evenly distributed over a first period of time, T₁, and any two neighboring pulse sets being separated by a second period of time, T₂; and adjusting the first period of time T₁ and the second period of time T₂ of the train of electrical pulses so that the efficacy of the train stimulation is identical to the optimal efficacy of the continuous stimulation of the target of interest.

In yet another aspect, the present invention relates to a system for stimulating a target of interest of a living subject with reduction of power consumption. In one embodiment, the system has a power supply; an IPG operably coupled with the power supply and configured to a train of electrical pulses, where the train of electrical pulses comprises a series of pulse sets, each of the plurality of pulse sets having a plurality of pulses time-evenly distributed over a first period of time, T₁, any two neighboring pulse sets of the series of pulse sets being separated by a second period of time, T₂, and any two neighboring pulses of the plurality of pulses being separated by a third period of time, T₃, and wherein T₁ and T₂ are in the order of milliseconds, and wherein T₁>T₃ and T₂≧T₃; and at least one electrode placed in the target of interest and operably coupled with the IPG for delivering the train of electrical pulses to a target of interest of a living subject for stimulation. The plurality of pulses is characterized with a pulse width, τ, an amplitude, H, and a frequency, f=1/T, wherein T=τ+T₃ being a pulse period, and wherein the frequency f is in the range of about 2-1000 Hz.

The system further has a controller being operable to cause the IPG to generate the train of electrical pulses.

In a further aspect, the present invention relates to a method for stimulating a target of interest of a living subject with reduction of power consumption. In one embodiment, the method comprises the steps of (a) delivering a plurality of pulses to the target of interest in a substantially repeating pattern for a first period of time, T₁, which is immediately followed by a second period of time, T₂, during which no pulses are delivered to the target of interest, wherein the plurality of pulses is delivered such that any two neighboring pulses of the plurality of pulses are occurred in a third period of time, T₃; and repeating step (a) for a predetermined times, wherein T₁ and T₂ are in the order of milliseconds, and wherein T₁>T₃ and T₂≧T₃.

The stimulating is performed with a stimulation device implanted in the living subject, wherein the stimulation device has an IPG for generating the plurality of pulses, a power supply adapted for powering the IPG, and at least one electrode placed in the target of interest and operably coupled with the IPG for delivering the plurality of pulses to the target of interest.

In yet a further aspect, the present invention relates to a system for stimulating a target of interest of a living subject. In one embodiment, the system has at least one implantable stimulation device having an IPG for generating a plurality of pulses, a power supply adapted for powering the IPG, and at least one electrode to be placed in the target of interest and operably coupled with the IPG for delivering the plurality of pulses to the target of interest.

The system also has a controller in communication with the at least one implantable stimulation device such that in operation, the controller and the at least one implantable stimulation perform the steps of delivering the plurality of pulses to the target of interest in a substantially repeating pattern for a first period of time, T₁, which is immediately followed by a second period of time, T₂, during which no pulses are delivered to the target of interest, wherein the plurality of pulses is delivered such that any two neighboring pulses of the plurality of pulses are occurred in a third period of time, T₃, and wherein T₁ and T₂ are in the order of milliseconds, and wherein T₁>T₃ and T₂≧T₃; and repeating the delivering step for a predetermined times.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 shows (A) a chart of a plurality of pulses in a substantially repeating pattern, and (B) a chart of a train of pulses according to one embodiment of the present invention.

FIG. 2 shows schematically a stereotactic and electrode placing system for a DBS implantation.

FIG. 3 shows schematically a diagram of a VIM stimulation.

FIG. 4 shows schematically a diagram of a STN stimulation.

### DETAILED DESCRIPTION OF THE PREFERRED EMBODIMENTS

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the term “living subject” refers to a human being such as a patient, or an animal such as a lab testing rat, monkey or the like.

As used herein, “target” refers to an object of stimulation in a deep brain of a living subject for treatment of a brain-controlled disorder, or in other anatomical regions of the living subject for treatment of other related disorders.

As used herein, “stimulation” refers to increase temporarily the activity of a body organ or part thereof responsive to an input signal to the body organ or part.

The term “place,” or “implant,” or “insert,” as used herein, is synonym in the specification and refers to put or embed a stimulation device, such as a microelectrode recording lead, macrostimulation lead, and/or a deep brain stimulator, into a target region of the body of a living subject.

### OVERVIEW OF THE INVENTION

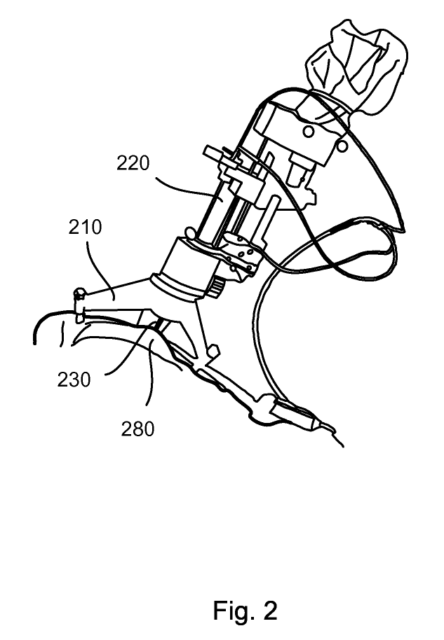

Electrical stimulation of an anatomical region of a patient through a stimulation device implanted in the anatomic region has gained a great deal of clinic relevance in treatment of certain disorders for the patent. However, the implantation of such a stimulation device into an anatomical region of a patient involves very sophisticated, time-consuming and costly surgical procedures. For example, for a typical implantation of a deep brain stimulator in the DBS, (i) a surgical plan is made based on preoperatively acquired images from the patient, which selects an initial target of stimulation; (ii) a customized stereotactic platform 210, as shown in FIG. 2, is manufactured based on the surgical plan, shipped to the hospital within a certain time frame and secured onto a target region of the skull 280 of the patient for mounting a micro-positioning drive 220; (iii) a burr hole is drilled on the skull 280; (iv) a microelectrode recording lead 230 is placed into the patient at the selected initial target position through the guide tube of the micro-positioning drive 220 attached to the platform 210; (v) a final target of stimulation is found by adjusting the position of the microelectrode recording lead 230 so that resting firing frequencies are noted or detected; (vi) the microelectrode lead is removed and a unipolar macrostimulation lead is inserted to the adjusted position as determined by the microelectrode recordings; (vii) with the patient awake, response to stimulation generated from the macrostimulation lead is monitored as the position of the macrostimulation lead is further adjusted until optimal stimulation to the deep brain target is detected; (viii) when the final positions are selected, the macrostimulation lead is removed and a deep brain stimulator lead is inserted at the final position; (ix) the proximal end of the DBS lead is then anchored to the skull and buried beneath the scalp; (x) the platform is then removed; (xi) within twenty-four hours of surgery, the imaging markers are re-attached to the posts and a post-operative CT scan is acquired; (xii) within about two weeks the patient is brought back to the operating room and the DBS lead is attached to an IPG, for example, Soletra (Medtronic, Inc., Minneapolis, Minn.), under general anesthesia; and (xiii) programming of the internal pulse generators is performed typically as an outpatient one month later by a neurologist.

The IPG is usually powered by a battery that is implanted with the IPG. The lifetime of the battery is about three to five years. In other words, additional surgery needs being conducted every three to five years for replacing the battery. Thus, it would gain a great deal of interest if the lifetime of the battery of a stimulation device could be prolonged without compromising the efficacy of the stimulation.

The present invention provides, among other things, methods and systems that utilize a train stimulation of a target of interest of a living subject for reduction of power consumption, thereby prolonging the lifetime of a power supply (battery) of a stimulation device in electrical stimulations.

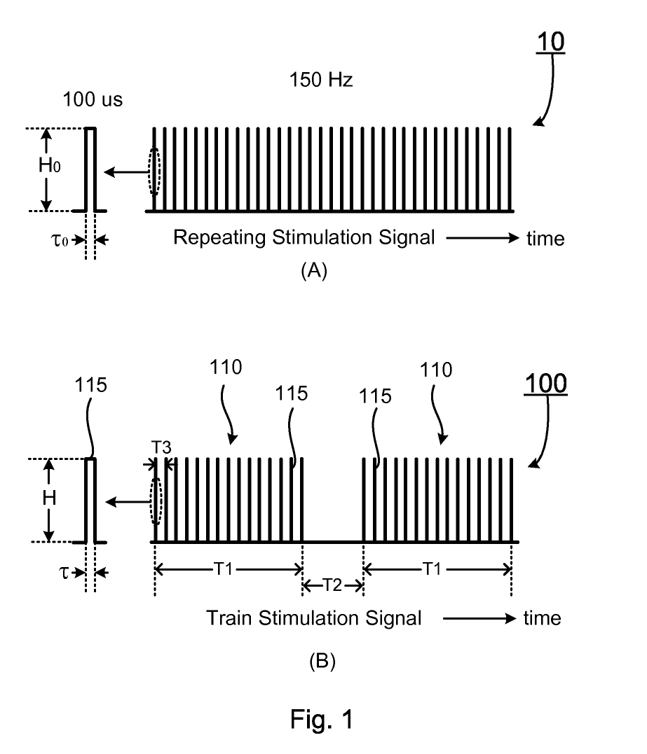

The description will be made as to the embodiments of the present invention in conjunction with the accompanying drawings of FIGS. 1-4. In accordance with the purposes of this invention, as embodied and broadly described herein, this invention, in one aspect, relates to a method for stimulating a target of interest of a living subject with reduction of power consumption. The target of interest of the living subject is corresponding to the STN, the VIM of the thalamus of the brain, or other anatomical regions of the living subject.

The stimulation is performed with a stimulation device implanted in the target region of the living subject. The stimulation device includes an IPG, a power supply adapted for powering the IPG, and one or more electrodes placed in the target of interest and operably coupled with the IPG.

The IPG is configured to generate a train of electrical pulses. Referring to FIG. 1B, the train of electrical pulses 100 includes a series of pulse sets 110. Each of the plurality of pulse sets 110 has a plurality of pulses 115 time-evenly distributed over a first period of time, T₁. Any two neighboring pulse sets of the series of pulse sets 110 are separated by a second period of time, T₂. Any two neighboring pulses of the plurality of pulses 115 are separated by a third period of time, T₃. The plurality of pulses 110 is characterized with a pulse width, τ, an amplitude, H, and a frequency, f=1/T, where T=τ+T₃ being a pulse period.

In one embodiment, the frequency f is in the range of about 2-1000 Hz. The first period of time T₁ and the second period of time T₂ are in the order of milliseconds, and T₁>T₃ and T₂≧T₃. When T₂=T₃, the train of pulses 100 is corresponding to an electrical signal of pulses in a substantially repeating pattern as shown in FIG. 1A. In one embodiment, 0.3<(T₂/T₁)<0.8. The first period of time T₁ is in the range of about 80-120 ms, and the second period of time T₂ is in the range of about 30-50 ms. For the train of pulses 100 shown in FIG. 1B, the first period of time T₁=100 ms, the second period of time T₂=42 ms, the pulse width τ=100 μs, the amplitude H=3 V, and the frequency f=150 Hz.

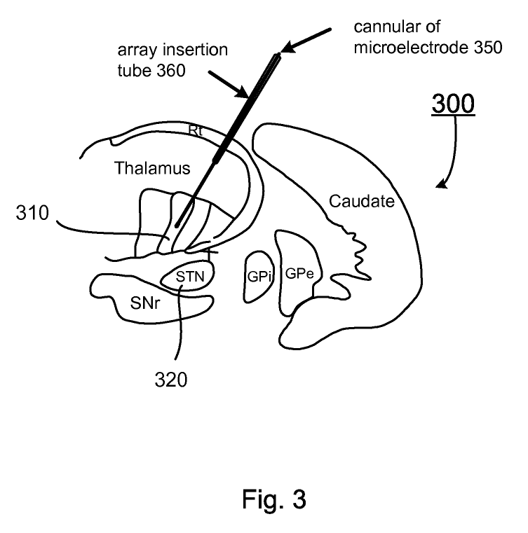

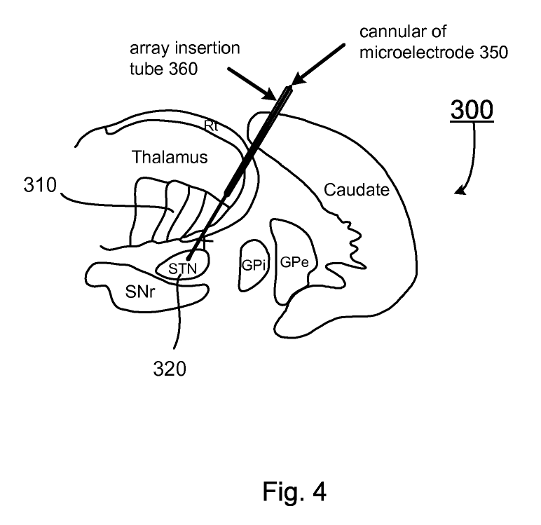

The train of pulses 100 is delivered by one or more electrodes to the target of interest. In exemplary embodiments, as shown in FIGS. 3 and 4, the target of interest is corresponding to the VIM 310 of the thalamus and the STN 320, respectively, of the brain 300 of a patient. The electrode 350 is placed through an array insertion tube 360 in the VIM 310 of the thalamus shown in FIG. 3 for the VIM stimulation, or in the STN 320 as shown in FIG. 4 for the STN stimulation.

The stimulation device may have a controller being operable to cause the IPG to generate the train of electrical pulses.

Additionally, the pulse width τ, the amplitude H, and the frequency f of the plurality of pulses, the first period of time T₁ and the second period of time T₂ of the train of pulses are determined such that when the train of pulses is delivered to the target of interest, the efficacy of stimulation by the train of pulses is identical to the optimal efficacy of stimulation by a standard stimulation signal of continuous pulses. This is obtained by the following procedures: at first, an electrical signal having pulses in a substantially repeating pattern, as shown in FIG. 1A, is delivered to the target of interest for a continuous stimulation of the target of interest. The electrical signal 10 is characterized with a pulse width, τ₀, an amplitude, H₀, and a frequency, f₀. Then, the pulse width τ₀, the amplitude H₀, and the frequency f₀ of the electrical signal 10 are adjusted so that an optimal efficacy of the continuous stimulation of the target of interest is obtained. The efficacy of stimulation of a target of interest is associated with improvements of related symptoms due to the stimulation. Next, a train of electrical pulses, as shown in FIG. 1B, is delivered to the target of interest for a train stimulation of the target of interest. The train of electrical pulses 100 comprises a series of pulse sets 110. Each pulse sets 110 has a plurality of pulses 115 with a pulse width τ=τ₀, an amplitude H=H₀, and a frequency f=f₀. The plurality of pulses 115 is time-evenly distributed over the first period of time, T₁. Additionally, any two neighboring pulse sets 110 are separated by the second period of time, T₂. Finally, the first period of time T₁ and the second period of time T₂ of the train of electrical pulses 100 are adjusted so that the efficacy of the train stimulation is identical to the optimal efficacy of the continuous stimulation of the target of interest.

For such a stimulation of the train of pulses, the lifetime of the battery (power supply) of the stimulation device can be prolonged.

One aspect of the present invention provides a method for stimulating a target of interest of a living subject with reduction of power consumption. The method, in one embodiment, includes delivering a plurality of pulses to the target of interest in a substantially repeating pattern for a first period of time, T₁, which is immediately followed by a second period of time, T₂, during which no pulses are delivered to the target of interest, wherein the plurality of pulses is delivered such that any two neighboring pulses of the plurality of pulses are occurred in a third period of time, T₃; and repeating the delivering step for a predetermined times. The first period of time T₁ and the second period of time T₂ are in the order of milliseconds with T₁>T₃ and T₂≧T₃.

Another aspect of the present invention provides a system for stimulating a target of interest of a living subject with reduction of power consumption. In one embodiment, the system has at least one implantable stimulation device having an IPG for generating a plurality of pulses, a power supply adapted for powering the IPG, and at least one electrode to be placed in the target of interest and operably coupled with the IPG for delivering the plurality of pulses to the target of interest. The system is configured to deliver a plurality of pulses to the target of interest in a substantially repeating pattern for a first period of time, T₁, which is immediately followed by a second period of time, T₂, during which no pulses are delivered to the target of interest, where the plurality of pulses is delivered such that any two neighboring pulses of the plurality of pulses are occurred in a third period of time, T₃. The delivering step is repeated for a predetermined times.

These and other aspects of the present invention are more specifically described below.

### IMPLEMENTATIONS AND EXAMPLES OF THE INVENTION

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Train Stimulation Having Identical Efficacy as Continuous Stimulation in VIM DBS

Deep brain stimulation of the ventralis intermedius nucleus of the thalamus of the brain of a patient is an effective and reversible therapy for medically refractory essential tremor. However, DBS implants are limited by battery life requiring additional surgery every three to five years. Current standard DBS therapy uses continuous stimulation at high frequency with variable pulse width and amplitude. According to the present invention, train stimulation with gaps of off-time between pulses prolongs the battery life of an internal pulse generator. Data from pain modulation and cortical mapping also indicates that train stimuli would be more dynamic and might prevent over-stimulation. The exemplary experiment was carried out to test the efficacy of a train stimulation on tremor reduction on one essential tremor patient during bilateral DBS implantation, as shown in FIG. 2.

Methods: As shown in FIG. 3, an intraoperative VIM mapping was performed using continuous stimulation via the macroelectrode (cannula tip of the microelectrode, FHC Inc, 1×0.28 mm exposure, 2500-3000Ω) connected to a Grass S-88 stimulator (not shown). Once the optimal target was located, the effect of continuous stimulation was compared to several sets of train stimulation with varying numbers of pulses per second (PPS). Identical monopolar stimulation parameters within each pulse having a frequency of about 150 Hz, a pulse width of about 150 μs, and an amplitude in the range of about 1-5 V were used. As shown in FIG. 1A, the continuous stimulation signal includes about 10 PPS with 100 ms pulse duration. As shown in FIG. 1B, the train stimulation signal includes about 6 to 10 PPS with 100 ms pulse duration. All stimulation modalities were generated by the Grass S-88 stimulator. The degree of tremor reduction was evaluated by a neurologist who was blinded to the type of stimulation.

Results: In two patients, seven PPS train stimulation produced the same degree of tremor reduction as the continuous stimulation at the same voltage. However, the train stimulation with PPS less than 7 showed diminished efficacy. Seven PPS is equivalent to cycling the 150 Hz stimulation on for 100 ms and off for 42 ms.

Observations: The preliminary data show that the efficacy of the train stimulation at 7 PPS was identical to the standard continuous stimulation at the same frequency, pulse width, and voltage. In principal, if the train stimulation at 7 PPS is used, up to 30% of implantable battery energy can be saved. The observations also indicate that the stimulation evoked effects last 42 ms following 100 ms of high frequency stimulation.

### Example 2

### Improved Energy Efficiency in Train Stimulation VS Continuous Stimulation of STN for Rigidity Suppression in a PD Patient

The exemplary experiment was carried out to test the efficacy of train stimulation on rigidity reduction on one Parkinson's disease patient during bilateral DBS implantation.

Methods: As shown in FIG. 4, an intraoperative STN mapping was performed involving continuous semi-microstimulation with a signal having a frequency of about 150 Hz, a pulse width of about 150 μs, and an amplitude in the range of about 1-5 V, generated by a Grass S-88 stimulator (not shown). Once an optimal response was located, the effect of continuous stimulation was compared to several sets of train stimulation with varying numbers of PPS. As shown in FIG. 1A, the continuous stimulation signal includes about 10 PPS with 100 ms pulse duration. As shown in FIG. 1B, the train stimulation signal includes about 6 to 10 PPS with 100 ms pulse duration. All stimulation modalities were generated by the Grass S-88 stimulator. The degree of rigidity reduction was evaluated by a neurologist blinded to the type of stimulation.

Results: Seven PPS train stimulation produced the same degree of rigidity suppression as continuous stimulation at the same voltage. However, train stimulation with PPS less than 7 showed diminished efficacy. Seven PPS is equivalent to cycling the 150 Hz stimulation on for 100 ms and off for 42 ms.

Observations: The preliminary data show that the efficacy of train stimulation at 7 PPS was identical to the standard continuous stimulation at the same frequency, pulse width, and voltage. If train stimulation at 7 PPS is used, up to 30% of implantable battery energy can be saved. This effect also provides evidence that high frequency STN stimulation has a prolonged effect, namely, 42 ms following 100 ms of high frequency stimulation.

The present invention provides, among other things, methods and systems that utilize a train stimulation of a target of interest of a living subject for reduction of power consumption, thereby prolonging the lifetime of a power supply (battery) of a stimulation device in electrical stimulations.

The foregoing description of the exemplary embodiments of the invention has been presented only for the purposes of illustration and description and is not intended to be exhaustive or to limit the invention to the precise forms disclosed. Many modifications and variations are possible in light of the above teaching.

These and other aspects of the present invention will become apparent from the following description of the preferred embodiment taken in conjunction with the drawings, given in the form of several appendices, although variations and modifications therein may be affected without departing from the spirit and scope of the novel concepts of the disclosure. Each and every of Appendices A and B is incorporated herein by reference in its entirety as an integral part of the application.

### LIST OF REFERENCES

[1]. Referen G. Deuschl, J. Volkmann, and P. Krack, “Deep brain stimulation for movement disorders”, Movement Disorders, vol. 17 (supplement 3), pp S1-S1, 2002.
[2]. B. Schrader, W. Hamel, D. Weinert, and H. M. Mehdorn, “Documentation of electrode localization.” Movement Disorders, vol. 17 (supplement 3), pp S167-S174, 2002.

What is claimed is:

1. A method for reducing power consumption in an implantable stimulation device having an internal pulse generator (IPG), a power supply adapted for powering the IPG, and at least one electrode operably coupled with the IPG, comprising the steps of:
a. causing the IPG to generate a train of electrical pulses, wherein the train of electrical pulses comprises a series of pulse sets, each of the plurality of pulse sets having a plurality of pulses time-evenly distributed over a first period of time, T₁, any two neighboring pulse sets of the series of pulse sets being separated by a second period of time, T₂, and any two neighboring pulses of the plurality of pulses being separated by a third period of time, T₃; and
b. delivering the train of electrical pulses to a target of interest of a living subject for stimulation by the at least one electrode placed in the target of interest.

2. The method of claim 1, wherein the plurality of pulses is characterized with a pulse width, τ, an amplitude, H, and a frequency, f=1/T, wherein T=τ+T₃ being a pulse period.

3. The method of claim 2, wherein the frequency f is in the range of about 2-1000 Hz.

4. The method of claim 2, further comprising the step of determining the pulse width τ, the amplitude H, and the frequency f of the plurality of pulses, the first period of time T₁ and the second period of time T₂.

5. The method of claim 4, wherein the determining step comprises the steps of:
a. delivering an electrical signal having pulses in a substantially repeating pattern to the target of interest for a continuous stimulation of the target of interest, wherein the electrical signal is characterized with a pulse width, τ₀, an amplitude, H₀, and a frequency, f₀;
b. adjusting the pulse width τ₀, the amplitude H₀, and the frequency f₀ of the electrical signal so that an optimal efficacy of the continuous stimulation of the target of interest is obtained;
c. delivering a train of electrical pulses to the target of interest for a train stimulation of the target of interest, wherein the train of electrical pulses comprises a series of pulse sets, each pulse sets having a plurality of pulses with a pulse width τ=τ₀, an amplitude H=H₀, and a frequency f=f₀, time-evenly distributed over a first period of time, T₁, and any two neighboring pulse sets being separated by a second period of time, T₂; and
d. adjusting the first period of time T₁ and the second period of time T₂ of the train of electrical pulses so that the efficacy of the train stimulation is identical to the optimal efficacy of the continuous stimulation of the target of interest.

6. The method of claim 1, wherein the first period of time T₁ and the second period of time T₂ of the train of electrical pulses are in the order of milliseconds, and wherein T₁>T₃ and T₂≧T₃.

7. The method of claim 6, wherein 0.3<(T₂/T₁)<0.8.

8. The method of claim 1, wherein the target of interest of the living subject is corresponding to the ventralis intermedius nucleus (VIM) of the thalamus, or the subthalamic nucleus (STN) of the brain of the living subject.

9. The method of claim 8, wherein the first period of time T₁ is in the range of about 80-120 ms, and the second period of time T₂ is in the range of about 30-50 ms.

10. The method of claim 1, wherein the implantable stimulation device further comprises a controller being operable to cause the IPG to generate the train of electrical pulses.

11. A method for stimulating a target of interest of a living subject with a stimulation device implanted therein, the stimulation device having an internal pulse generator (IPG), a power supply adapted for powering the IPG, and at least one electrode placed in the target of interest and operably coupled with the IPG, comprising the steps of:
a. causing the IPG to generate a train of electrical pulses, wherein the train of electrical pulses comprises a series of pulse sets, each of the plurality of pulse sets having a plurality of pulses time-evenly distributed over a first period of time, T₁, any two neighboring pulse sets of the series of pulse sets being separated by a second period of time, T₂, and any two neighboring pulses of the plurality of pulses being separated by a third period of time, T₃; and
b. delivering the train of electrical pulses to a target of interest of a living subject for stimulation by the at least one electrode.

12. The method of claim 11, wherein the plurality of pulses is characterized with a pulse width, τ, an amplitude, H, and a frequency, f=1/T, wherein T=τ+T₃ being a pulse period.

13. The method of claim 12 wherein the frequency f is in the range of about 2-1000 Hz.

14. The method of claim 12, further comprising the step of determining the pulse width τ, the amplitude H, and the frequency f of the plurality of pulses, the first period of time T₁ and the second period of time T₂.

15. The method of claim 14, wherein the determining step comprises the steps of:
a. delivering an electrical signal having pulses in a substantially repeating pattern to the target of interest for a continuous stimulation of the target of interest, wherein the electrical signal is characterized with a pulse width, τ₀, an amplitude, H₀, and a frequency, f₀;
b. adjusting the pulse width τ₀, the amplitude H₀, and the frequency f₀ of the electrical signal so that an optimal efficacy of the continuous stimulation of the target of interest is obtained;
c. delivering a train of electrical pulses to the target of interest for a train stimulation of the target of interest, wherein the train of electrical pulses comprises a series of pulse sets, each pulse sets having a plurality of pulses with a pulse width τ=τ₀, an amplitude H=H₀, and a frequency f=f₀, time-evenly distributed over a first period of time, T₁, and any two neighboring pulse sets being separated by a second period of time, T₂; and
d. adjusting the first period of time T₁ and the second period of time T₂ of the train of electrical pulses so that the efficacy of the train stimulation is identical to the optimal efficacy of the continuous stimulation of the target of interest.

16. The method of claim 11, wherein the first period of time T₁ and the second period of time T₂ of the train of electrical pulses are in the order of milliseconds, and wherein T₁>T₃ and T₂≧T₃.

17. The method of claim 11, wherein the target of interest of the living subject is corresponding to the ventralis intermedius nucleus (VIM) of the thalamus, or the subthalamic nucleus (STN) of the brain of the living subject.

18. The method of claim 17, wherein the first period of time T₁ is in the range of about 80-120 ms, and the second period of time T₂ is in the range of about 30-50 ms.

19. The method of claim 11, wherein the implantable stimulation device further comprises a controller being operable to cause the IPG to generate the train of electrical pulses.

20. A system for stimulating a target of interest of a living subject with reduction of power consumption, comprising:
a. a power supply;
b. an internal pulse generator (IPG) operably coupled with the power supply and configured to a train of electrical pulses, wherein the train of electrical pulses comprises a series of pulse sets, each of the plurality of pulse sets having a plurality of pulses time-evenly distributed over a first period of time, T₁, any two neighboring pulse sets of the series of pulse sets being separated by a second period of time, T₂, and any two neighboring pulses of the plurality of pulses being separated by a third period of time, T₃, and wherein T₁ and T₂ are in the order of milliseconds, and wherein T₁>T₃ and T₂≧T₃; and
c. at least one electrode placed in the target of interest and operably coupled with the IPG for delivering the train of electrical pulses to the target of interest of the living subject for stimulation.

21. The system of claim 20, further comprising a controller being operable to cause the IPG to generate the train of electrical pulses.

22. The system of claim 20, wherein the plurality of pulses is characterized with a pulse width, τ, an amplitude, H, and a frequency, f=1/T, wherein T=τ+T₃ being a pulse period, and wherein the frequency f is in the range of about 2-1000 Hz.

23. A method for stimulating a target of interest of a living subject with reduction of power consumption, comprising the steps of:
a. delivering a plurality of pulses to the target of interest in a substantially repeating pattern for a first period of time, T₁, which is immediately followed by a second period of time, T₂, during which no pulses are delivered to the target of interest, wherein the plurality of pulses is delivered such that any two neighboring pulses of the plurality of pulses are occurred in a third period of time, T₃; and
b. repeating step (a) for a predetermined times,
wherein T₁ and T₂ are in the order of milliseconds, and wherein T₁>T₃ and T₂≧T₃.

24. The method of claim 23, wherein the stimulating is performed with a stimulation device implanted in the living subject, wherein the stimulation device has an internal pulse generator (IPG) for generating the plurality of pulses, a power supply adapted for powering the IPG, and at least one electrode placed in the target of interest and operably coupled with the IPG for delivering the plurality of pulses to the target of interest.

25. A system for stimulating a target of interest of a living subject, comprising:
a. at least one implantable stimulation device having an internal pulse generator (IPG) for generating a plurality of pulses, a power supply adapted for powering the IPG, and at least one electrode to be placed in the target of interest and operably coupled with the IPG for delivering the plurality of pulses to the target of interest; and
b. a controller in communication with the at least one implantable stimulation device such that in operation, the controller and the at least one implantable stimulation perform the steps of:
(i). delivering the plurality of pulses to the target of interest in a substantially repeating pattern for a first period of time, T₁, which is immediately followed by a second period of time, T₂, during which no pulses are delivered to the target of interest, wherein the plurality of pulses is delivered such that any two neighboring pulses of the plurality of pulses are occurred in a third period of time, T₃, and wherein T₁ and T₂ are in the order of milliseconds, and wherein T₁>T₃ and T₂≧T₃; and
(ii). repeating step (a) for a predetermined times.
